(19) United States
(12) Patent Application Publication
(10) Pub. No.: US 2020/0248201 A1
(43) Pub. Date: Aug. 6, 2020

(54) IMPROVED YIELD IN PLANTS BY OVEREXPRESSING A TREHALOSE-6 PHOSPHATE SYNTHASE

(71) Applicant: BIOGEMMA, Paris (FR)

(72) Inventor: Elise REDONDO, Greer, SC (US)

(73) Assignee: BIOGEMMA, Paris (FR)

(21) Appl. No.: 16/652,130

(22) PCT Filed: Oct. 4, 2018
(86) PCT No.: PCT/EP2018/076981

§ 371 Date: Mar. 30, 2020

(30) Foreign Application Priority Data
Oct. 5, 2017 (EP) 17306338.9

(51) Int. Cl. C12N 15/82; C12N 9/10

(52) U.S. Cl. CPC C12N 15/8273; C12N 9/1051; C12N 15/8205; C12N 15/8216; C12Y 204/01015

(57) ABSTRACT

The present invention relates to a method for improving yield in plants by overexpressing a class II threalose-6 phosphatase or a fragment thereof. Also, the present invention is related to a method for identifying said plants with improved yield and a method of growing said plants. A construct comprising a nucleic acid encoding said class II threalose-6 phosphatase and transgenic plants comprising said construct are other aspects of the present invention.

The invention relates to the field of plant improvement, in particular of the improvement of yield for plants. In particular, the present invention relates to a method for improving yield in plants by overexpressing a class II threhalose-6 phosphate synthase or a fragment thereof. Also, the present invention is related to a method for identifying said plants with improved yield and a method of growing said plants. A construct comprising a nucleic acid encoding said class II threhalose-6 phosphate synthase and transgenic plants comprising said construct are other aspects of the present invention.

### BACKGROUND

In agriculture, yield is the amount of product harvested from a given acreage (eg weight of seeds per unit area). It is often expressed in metric quintals (1 q=100 kg) per hectare in the case of cereals. It is becoming increasingly important to improve the yield of seed crops to feed an expanding world population. One strategy to increase the yield is to increase the seed size, provided that there is not a concomitant decrease in seed number.

Another important issue to be addressed to respond to today's agricultural challenges is obtaining plants capable of maintaining or increasing yield under stress conditions compared to normal conditions. More and more farmers worldwide are affected by drought stress that can greatly impair plant development growth and ultimately yield.

Drought stress, or water deficit, occurs when water supply in the soil is reduced and/or water loss by transpiration or evaporation occurs continuously. When drought stress intensity is strong, it is called desiccation.

Trehalose (α-D-glucopyranosyl α-D-glucopyranoside) is a non-reducing disaccharide ubiquitously found in bacteria, archaea, fungi or invertebrates where it functions as a compatible solute, osmoprotectant (in bacteria, fungi and invertebrates) or carbon reserve. In few resurrection plants, trehalose has been detected in relative large amount while most higher plants accumulate only traces amount of trehalose (Leyman et al., 2001). Accordingly, trehalose pathway is widespread, and at least five biosynthetic pathways evolved since bacteria. In plants, as in yeast, a two-step reaction occurs with synthesis of trehalose-6-phosphate (T6P) from UDP-glucose and glucose-6-phosphate catalyzed by the trehalose-6 phosphate synthase (TPS). Subsequently, a dephosphorylation of T6P to trehalose is catalyzed by the trehalose-6-phosphate phosphatase (TPP). Catabolism of trehalose is taken over by the trehalase which triggers hydrolysis to glucose. Both TPS and TPP proteins are encoded by multi-gene families while the trehalase is usually found at a single copy level in plant genomes (Lunn, 2007).

Plants with altered expression of the trehalose pathway genes show a large range of phenotypes, including effects on embryogenesis, vegetative growth, flowering, abiotic and biotic stress tolerance (Lunn et al., 2014) supporting the hypothesis that trehalose pathway play important roles in plant metabolism and development.

Plant TPS proteins are encoded by multi-gene families, with Arabidopsis and rice genomes encoding both for 11 TPS genes while 14 TPS genes has been found in maize. Because the wheat genome is not yet fully available, the number of TPS genes may exceed 12 genes (Xie et al., 2015). As previously described (Yang et al., 2012; Henry et al., 2014), the TPS gene family is divided into two classes encoding class I TPS and class II TPS proteins. This dichotomy appeared early in the green lineage and is found in both monocot and dicots. Surprisingly, class I and class II TPS genes show distinct characteristics in copy number, gene expression patterns, and gene structure. All class I genes from Populus, Arabidopsis, rice or maize have 16 introns while class II genes contain much fewer introns, usually only 2 introns are retained (Yang et al., 2012). This strict conservation of the TPS gene structure suggests the TPS gene functions evolved independently between class I and class II genes.

Class I and class II plant TPS proteins contain both a TPS and a TPP domain (Yang et al., 2012). All Arabidopsis class I TPS, except AtTPS3 which is likely encoded by a pseudo-gene, and the rice OsTPS1 has been shown to have TPS activity by yeast complementation of the mutant Δtps1 (by AtTPS1) or Δtps1Δtps2 double mutant (by AtTPS1, 2 or 4) (Vandesteene et al., 2010; Zang et al., 2011; Delorge et al., 2015). At the opposite, no Class II TPS protein was shown to have catalytic activity so far. However, 2 rice class II TPS proteins were shown interacting with the catalytically active class I TPS into high molecular weight complexes in vitro (Zang et al., 2011). Nevertheless class II TPS proteins may still bind their substrate G6P. For instance the pathogenic fungi Magnaporthe grisea TPS involves G6P binding without formation of T6P (Wilson et al., 2007). Thus class II TPS seem to have lost their enzymatic activity but would rather sense the level of trehalose pathway activity (Henry et al., 2014). Through their interaction with catalytically active class I TPS, class II TPS may contribute to the regulation of T6P level for plant carbohydrate sensing.

The maize genome encodes for 2 class I TPS and 12 class II TPS based on protein sequence phylogeny (Henry et al., 2014). All maize class II TPS displayed a substitution of arginine to aspartic acid in the UDP-glucose phosphate binding domain which may strongly affect enzymatic activity (Henry et al., 2014).

While the over-expression of the rice OsTpp1 gene in maize ear sustain maize yield under water-deficit condition (Nuccio et al., 2015), no TPS engineering have been demonstrated to provide such yield improvement in crop so far. Toward this goal, some preliminary results have been reported. Several studies reports induced expression or increased activity of TPS enzymes under abiotic stresses in cotton (Kosmas et al., 2006), in maize (Jiang et al., 2010), in rice (Li et al., 2011), in cassava (Han et al., 2016) or in the xerophytic plant Capparis ovata (Ilhan et al., 2015). In winter wheat, some TPS genes have been shown to be induced by freezing (Xie et al., 2015). Over-expression of the catalytically active rice OsTPS1 improve tolerance to abiotic stress in rice plantlets (Li et al., 2011). Other TPS was engineered to improve photosynthetic performance under high light conditions in the alga Parachlorella kessleri (Rathod et al., 2016) and to protect seeds under chilling stress (Wang 2016). To our knowledge, the role of class II TPS in crop remains elusive and their role in yield maintenance under normal or stress conditions has not yet been reported.

The sequence of a maize class II TPS is disclosed in US20090170173 but the applicants did not establish a link between this sequence and an improvement of yield or drought tolerance in transformed crops. The sequence was merely cited amongst hundreds of other sequences and linked to lipid and sugar metabolisms.

The sequence of another maize class II TPS is disclosed in US20120266327 amongst hundreds of other sequences. This sequence is merely cited in the sequence listing. The applicants focused on a fusion of TPS and TPP to improve crops.

In US20130045323 and US20130045324, the applicants tested several Arabidopsis TPS from class II in maize. Their initial purpose was to increase the protein, oil and amino acid content in seeds. They observed no significant decrease in yield. These applications do not show an involvement of TPS7 from class II in neither yield nor drought tolerance.

In EP0901527, the patent is dealing with the manipulation of TPS and TPP in dicotyledonous plants. The maize class II TPS are not disclosed nor their involvement in drought tolerance and yield improvement.

U.S. Pat. No. 8,124,840 protects a number of phenotypes that can be improved by transforming a plant with a nucleic acid encoding a trehalose phosphate synthase. None of these phenotypes are yield improvement of drought tolerance. Moreover, according to the specification, this patent family deals with TPS from class I, TPS with an enzymatic activity.

There is still a need of developing plants, notably monocotyledons, with maintained or improved yield capacity measured in field conditions under normal or drought stress conditions.

### SUMMARY OF THE INVENTION

The present invention is related to a method for improving yield in plants, said method comprising overexpressing a class II TPS protein comprising at least one of the six following domains, preferably the six following domains:
Domain 1 as set forth in SEQ ID NO: 1: FCKQX₁LWPLFHYMLPX₂CX₃DKX₄ELFDRX₅LFX₆AYVRAN, wherein
X₁ can be Q or H
X₂ can be I or V
X₃ can be L or H
X₄ can be G or D
X₅ can be S or N or T
X₆ can be Q or R
Domain 2 as set forth in SEQ ID NO: 2: DDDX₇VWVHDYHLMLX₈PTX₉LRKX₁₀LHRIKX₁₁GFFLHSPFPSSEIYX₁₂X₁₃LPVRDEI LKSLLNADLIGFQTFDYARHFLSCCSRLLGLX₁₄YESKRGX₁₅IGIX₁₆YFGRTVX₁₇LKIL, wherein
X₇ can be F or C or H or Y
X₈ can be L or I or V
X₉ can be F or L
X₁₀ can be R or F
X₁₁ can be I or V or L
X₁₂ can be R or K
X₁₃ can be T or S
X₁₄ can be H or N
X₁₅ can be Y or H
X₁₆ can be E or D
X₁₇ can be S or N
Domain 3 as set forth in SEQ ID NO: 3: LGVDDMDIFKGISLKX₁₈LX₁₉LEX₂₀LLX₂₁RX₂₂PKLRX₂₃KVVLVQIX₂₄NPARSX₂₅GKD, wherein
X₁₈ can be F or L
X₁₉ can be G or A
X₂₀ can be L or F
X₂₁ can be D or E
X₂₂ can be N or T
X₂₃ can be E or G or Q
X₂₄ can be I or V
X₂₅ can be T or I or P
Domain 4 as set forth in SEQ ID NO: 4: AASDCCIVNAX₂₆RDGMNLX₂₇PYEYTVCRQGN, wherein
X₂₆ can be V or L
X₂₇ can be V or I
Domain 5 as set forth in SEQ ID NO: 5: HTSTLIVSEFVGCSPSLSGAFRVNPWSX₂₈X₂₉DVADAL, wherein
X₂₈ can be V or M or I
X₂₉ can be D or E
Domain 6 as set forth in SEQ ID NO: 6: RCWX₃₀X₃₁GFGLNFRX₃₂IALSPGFRX₃₃LSX₃₄EH, wherein
X₃₀ can be A or T
X₃₁ can be I or T
X₃₂ can be V or I
X₃₃ can be K or R
X₃₄ can be S or L;
   said protein to be overexpressed in the plant having at least 70% sequence identity with SEQ ID NO: 7.

The present invention is also related to a method to identify a plant with improved yield comprising the step of identifying in a population of plants, the plants overexpressing a protein comprising at least one of the six domains defined above, preferably the six domains, and having at least 70% sequence identity with SEQ ID NO: 7 or a protein comprising the six domains defined above.

Preferably said method to identify a plant with improved yield comprises identifying plants overexpressing the protein of sequence SEQ ID NO: 7 (TPS7_a) or SEQ ID NO: 8 (TPS7_b).

The present invention is related to a method of growing plants comprising the steps of:
(i) sowing plant seeds, wherein said plant seeds originate from plants overexpressing a protein comprising at least one of the six domains defined above as set forth in SEQ ID NO: 1 to 6, preferably the six domains, and having at least 70% sequence identity with SEQ ID NO: 7, preferably a protein of sequence SEQ ID NO: 7 or SEQ ID NO: 8, and
(ii) growing plants from these sowed seeds.

Preferably the methods according to the present invention is related to overexpressing the protein of sequence SEQ ID NO: 7 or SEQ ID NO: 8.

The present invention is related to a nucleic acid construct comprising a rab17 promoter operably linked to a nucleic acid sequence encoding a protein having at least 70% sequence identity with SEQ ID NO: 7, preferably encoding a protein having at least 92% sequence identity with SEQ ID NO: 7.

Another aspect of the present invention is also related to transgenic plants comprising said nucleic acid constructs defined above.

### DETAILED DESCRIPTION OF THE INVENTION

In a first aspect, the present invention is related to a method for improving yield in plants, said method comprising overexpressing a class II TPS protein comprising at least one of the six following domains:
Domain 1 as set forth in SEQ ID NO: 1: FCKQX₁LWPLFHYMLPX₂CX₃DKX₄ELFDRX₅LFX₆AYVRAN, wherein
X₁ can be Q or H
X₂ can be I or V
X₃ can be L or H
X₄ can be G or D
X₅ can be S or N or T
X₆ can be Q or R
Domain 2 as set forth in SEQ ID NO: 2: DDDX₇VWVHDYHLMLX₈PTX₉LRKX₁₀LHRIKX₁₁GFFLHSPFPSSEIYX₁₂X₁₃LPVRDEI LKSLLNADLIGFQTFDYARHFLSCCSRLLGLX₁₄YESKRGX₁₅IGIX₁₆YFGRTVX₁₇LKIL, wherein
X₇ can be F or C or H or Y
X₈ can be L or I or V
X₉ can be F or L
X₁₀ can be R or F
X₁₁ can be I or V or L
X₁₂ can be R or K
X₁₃ can be T or S
X₁₄ can be H or N
X₁₅ can be Y or H
X₁₆ can be E or D
X₁₇ can be S or N
Domain 3 as set forth in SEQ ID NO: 3: LGVDDMDIFKGISLKX₁₈LX₁₉LEX₂₀LLX₂₁RX₂₂PKLRX₂₃KVVLVQIX₂₄NPARSX₂₅GKD, wherein
X₁₈ can be F or L
X₁₉ can be G or A
X₂₀ can be L or F
X₂₁ can be D or E
X₂₂ can be N or T
X₂₃ can be E or G or Q
X₂₄ can be I or V
X₂₅ can be T or I or P
Domain 4 as set forth in SEQ ID NO: 4: AASDCCIVNAX₂₆RDGMNLX₂₇PYEYTVCRQGN, wherein
X₂₆ can be V or L
X₂₇ can be V or I
Domain 5 as set forth in SEQ ID NO: 5: HTSTLIVSEFVGCSPSLSGAFRVNPWSX₂₈X₂₉DVADAL, wherein
X₂₈ can be V or M or I
X₂₉ can be D or E
Domain 6 as set forth in SEQ ID NO: 6: RCWX₃₀X₃₁GFGLNFRX₃₂IALSPGFRX₃₃LSX₃₄EH, wherein
X₃₀ can be A or T
X₃₁ can be I or T
X₃₂ can be V or I
X₃₃ can be K or R
X₃₄ can be S or L;
   said protein having at least 70% sequence identity with SEQ ID NO: 7.

In the context of the present invention, the expression “to improve the yield” means that the yield of a plant that overexpress the class II TPS protein according to the present invention is increased compared to a plant that does not overexpress said class II TPS protein.

In one embodiment, the method for improving yield in plants according to the present invention comprises overexpression of a protein comprising at least one, at least two, at least three, at least four, at least five or comprising the six domains as defined above by SEQ ID NO: 1 to SEQ ID NO: 6, and having at least 70% sequence identity with SEQ ID NO: 7.

In a particular embodiment, the method for improving yield in plants of the invention comprises overexpression of a protein comprising the six domains as defined above by SEQ ID NO: 1 to SEQ ID NO: 6, and having at least 70% sequence identity with SEQ ID NO: 7.

In a more preferred embodiment, the protein to be overexpressed in plants for improving yield is a class II trehalose phosphate synthase as defined above and having a sequence of at least 92% sequence identity to SEQ ID NO: 7.

According to the present invention, “sequence identity” is defined by conducting a global optimal alignment over the whole length of the sequences, for example by using the algorithm of (Needleman & Wunsch, 1970), in particular with default parameters.

In a particular embodiment, the sequences with at least 70% sequence identity to SEQ ID NO: 7 may be selected in the group consisting of SEQ ID NO: 9 to SEQ ID NO: 16.

The most preferred embodiment is related to the overexpression in plants a protein of sequence SEQ ID NO: 7 or a protein of sequence SEQ ID NO: 8 for improving yield in plants.

Overexpression of the class II TPS as defined in the present invention for improving plant yield may carried out in any plants. As examples, it may be mentioned monocotyledons such as maize, wheat, sorgho, rice, barley, sugarcane, or dicotyledons such as sunflower, sugarbeet rapeseed, tomato, potato and the like.

Similarly, the class II TPS protein to be overexpressed in plants for improving yield according to the invention may be from any type of plants. For example, from Zea maize, Sorghum bicolor, Brachipodium distachyon, Setaria italica, Oryza sativa, and the like.

Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. The yield may be expressed for example in q/ha (q means quintal which correspond to 100 kg and ha means hectare).

For the present invention, the yield may be calculated as follows:
During harvest, grain weight and grain moisture are measured using on-board equipment on the combine harvester.
Grain weight is then normalized to moisture at 15%, using the following formula:

Normalized grain weight=measured grain weight×(100−measured moisture (as a percentage))/85 (which is 100−normalized moisture at 15%).

As an example, if the measured grain moisture is 25%, the normalized grain weight will be:

normalized grain weight=measured grain weight×75/85.

Yield is then expressed in a conventional unit (such as quintal per hectare).

The invention can be performed by any conventional methods for efficient overexpression in plants.

It may be obtained by direct mutation conducting to overexpression in the plant cell of the gene encoding the class II TPS as defined above according to the invention with gene editing techniques, such as CRISPR/Cas9 (WO2013181440) or TALEN.

Other techniques that may be used for overexpressing the protein defined in the present invention are also well known by the skilled person, such as transformation, particularly with a vector comprising a nucleic acid sequence encoding the protein to be overexpressed under the control of a promoter functional in plants. Said transformation may be performed with bacterial strains such as Agrobacterium tumefaciens or by direct methods such as electroporation, gene gun bombardment, direct precipitation by means of PEG or other method known by the person skilled in the art. Preferably, the transformation of a plant may be carried out with a vector comprising a nucleic acid sequence encoding the protein to be overexpressed under the control of a promoter functional in plants, said vector being introduced into the plant by Agrobacterium tumefaciens. In particular, it is possible to use the method described by Ishida et al. (Nature Biotechnology, 14, 745-750, 1996) for the transformation of Monocotyledons.

In a preferred embodiment, the method for improving yield in plants according to the present invention is carried out by transforming the plant with a vector comprising a promoter functional in plants and a nucleic acid sequence encoding the protein having at least one of the six domains defined above, of sequence as set forth in SEQ ID NO: 1 to SEQ ID NO: 6, preferably the six domains, and having at least 70%, preferably at least 92%, sequence identity with SEQ ID NO: 7.

More preferably, the vector to be used in the method of the invention comprises a promoter functional in plants and a nucleic acid sequence encoding the protein of SEQ ID NO: 7 or encoding the protein of SEQ ID NO: 8.

According to the present invention, a promoter “functional in plants” is a promoter that is able to drive expression of a gene operably linked thereto in a plant cell.

For being expressed, a sequence coding for the protein to be overexpressed as defined above, and preferably a protein as set forth in SEQ ID NO: 7 or in SEQ ID NO: 8, may be present under the control of a constitutive, tissue specific, developmentally regulated, inducible or meiosis promoter. Other suitable promoters could be used. It could be a tissue-specific promoter such as a leaf-specific promoter, a seed-specific, a BETL (Basal Endosperm Transfer Layer) specific promoter and the like. Numerous tissue-specific promoters are described in the literature and any one of them can be used. One can also cite the promoters regulated during seed development such as the HMWG promoter (High Molecular Weight Glutenin) of wheat (Anderson & Greene, 1989; Robert et al., 1989), the waxy, zein or bronze promoters of maize, or the promoters disclosed in US 20150007360, US 20120011621, US 20100306876, US 20090307795 or US 20070028327.

Promoters may come from the same species or from another species (heterologous promoters). Although some promoters may have the same pattern of regulation when there are used in different species, it is often preferable to use monocotyledonous promoters in monocotyledons and dicotyledonous promoters in dicotyledonous plants.

In a preferred embodiment, said vector comprises a promoter which is active in leaf tissues. A promoter active in leaf tissue can be a promoter which drives expression in leaf tissues but also drive expression in other tissues or it can be a promoter which drives expression specifically in leaf tissues with a residual activity in other tissues or it can be a promoter which drives expression specifically in leaf tissues and nowhere else.

Examples of promoters active in leaf tissues useful for expression include the phosphoenolypurate carboxylase promoter from sorgho (Crétin et al., 1991), Rubisco small subunit promoter (rbcS) (Matsuoka & Sanada, 1991), proOsCAB (Sugiyama et al., 2001), proZmCA (Matsuoka et al., 1994).

The rbcs promoter depicted as SEQ ID NO: 17 is a preferred promoter usable in the context of the present invention.

The rab17 promoter induced by drought and able to drive expression in leaf tissues depicted as SEQ ID NO: 18 is another preferred promoter usable in the context of the present invention.

The method for improving yield in plants is particularly useful and efficient under drought conditions or said differently, under drought stress. Improvement of the yield under drought stress means that the yield of a plant that overexpress the class II TPS protein as defined above is maintained compared to a plant cultivated under normal watering conditions.

As used herein, the term “drought stress” refers to a condition without normal watering in plant growth, which is utilized as a very common term including all kind of abiotic stresses that induce harmful effects on plant growth and survival, for example “drought stress” as used herein includes such stresses as e.g., soil water deficit, vapor pressure deficit, heat stress or light radiation. More specifically, the term “drought” refers to environmental conditions where the amount of water (e.g., rainfall or other available water source for plant life) is less than the average water conditions for the particular environment, or the amount of water available is less than the amount of water typically needed by a certain species of plant or by a plant growing in a particular environment.

According to the present application, a drought stressed location is a location where the grain yield potential of the site has not been reached due to a drought stress.

A non-stressed location is a location where the grain yield potential has been reached by a commercial hybrid variety.

The drought stress intensity is evaluated by measuring the yield lost between the drought stress treatment (WUE) and a reference treatment irrigated with an optimal amount of water, which is at least, equivalent to the maximum evapotranspiration (ETM) of the crop.

A yield loss of −30% is targeted with a common distribution of the drought location between −10% and −40% of yield.

A low drought stressed location is typically a location with a yield lost between 0% and up to −20%, a moderate stressed location between −20% and up to −30%.

The targeted growth stage period is typically from tasseling to R2 growth stage. In a common drought location, the drought stress period can spread out from a period between V10 and R4 growth stage.

The terms “drought-resistance” or “drought-tolerance” refer to the ability of a plant to recover from periods of drought stress (i.e., little or no water for a period of days). In the context of the present invention, drought tolerance refers to the ability of a plant to achieve a yield performance as close as possible to the optimal yield whatever the intensity and the duration of the stress.

In a second aspect, the present invention is related to a method to identify a plant with improved yield comprising the step of identifying in a population of plants, the plants overexpressing a protein comprising at least one of the six domains as defined above as set forth in SEQ ID NO: 1 to SEQ ID NO: 6, preferably the six domains, and having at least 70%, preferably at least 92%, sequence identity with SEQ ID NO: 7.

As above, in a preferred embodiment, this method comprises the step of identifying in a population of plants, the plants overexpressing a protein of sequence SEQ ID NO: 7 or of sequence SEQ ID NO: 8.

In a third aspect, the present invention is related to a method of growing plants comprising the steps of:
(i) sowing plant seeds, wherein said plant seeds originate from plants overexpressing a class II TPS protein comprising at least one of the six domains defined above as set forth in SEQ ID NO: 1 to SEQ ID NO: 6, preferably the six domains, and having at least 70%, preferably at least 92%, sequence identity with SEQ ID NO: 7, and
(ii) growing plants from these sowed seeds.

Similarly, in a preferred embodiment, this method comprises the step of sowing plant seeds which originate from plants overexpressing a protein of sequence SEQ ID NO: 7 or of sequence SEQ ID NO: 8.

In a preferred embodiment, the step of growing plants (ii) from the above defined sowed seeds is made under drought stress.

In a fourth aspect, the present invention is related to a nucleic acid construct comprising a rab17 promoter operably linked to a nucleic acid sequence encoding a class II TPS protein comprising at least one of the six domains defined above as set forth in SEQ ID NO: 1 to SEQ ID NO: 6, preferably the six domains, and having at least 70% sequence identity with SEQ ID NO: 7, or preferably and having at least 92% sequence identity with SEQ ID NO: 7.

More preferably, the nucleic acid construct according to the invention comprises a nucleic acid sequence encoding the protein of SEQ ID NO: 7 or encoding the protein of SEQ ID NO: 8.

Transgenic plants comprising the above defined nucleic acid construct in all the particular embodiment described, are another aspect of the present invention.

### EXAMPLES

### Example 1

### Association Studies

The aim of association studies is to identify loci contributing to quantitative traits, based on statistical association between genotypes and phenotypes using a large germplasm collection (panel) without knowledge on pedigree. At the opposite of linkage mapping, association studies can be performed using a selection of cultivars without the need for crossing and screening offspring. In this way, it can be looked at a maximum of genotypic variability (depending on panel selection) in a single study. Thus, using this technique, it is possible to identify favorable alleles of the TPS7_a and TPS7_b genes linked to phenotypic data, with a high resolution. A SNPs discovery has been done in the genes of interest (e.g. TPS7_a and TPS7_b), that are then linked to phenotypic data. Results expected are positive association between SNPs and phenotypic data to conclude on the implication of the gene in the QTL's effect. Linkage Disequilibrium in the area has to be considered. Association study can provide information on gene polymorphisms implicated in traits and can indicate which allele is favorable regarding these traits. In TPS7_a (chr1), 5 SNPs show significant association results between genotypic and phenotypic data on yield and tolerance to drought stress in several environments (different years, sites, plant treatments). In TPS7_b (chr4), one SNP shows significant association results between genotypic and phenotypic data on yield in several environments. Globally, it indicates a direct link between TPS7_a and TPS7_b with yield improvement in optimal conditions or under drought conditions with positive allele of these 2 genes.

### Example 2

### Cloning of TPS7_b Under the Rbcs Promoter and Transformation

The ZmTPS7_b coding sequence (SEQ ID NO: 20 encoding the protein sequence SEQ ID NO: 8) was codon optimized for maize expression by a gene synthesis service provider and cloned into the pUC57 vector (Genscript). The optimized ZmTPS7_b sequence was linked to the Rbcs promoter (Matsuoka & Sanada, 1991) (SEQ ID NO: 17) and a Zea mays Rbcs polyadenylation sequence (SEQ ID NO: 21), by performing a restriction enzyme digestion and ligation in the destination binary plasmid pBIOS03092 forming pBIOS03538, thus leading to the cassette of sequence SEQ ID NO: 23.

pBIOS03538 was transferred into agrobacteria LBA4404 (pSB1) according to Komari et al (Komari et al., 1996). Maize cultivar A188 was transformed with these agrobacterial strains essentially as described by Ishida et al (Ishida et al., 1996).

Analysis of the pRbcs-TPS7_b transformed corn plants indicated that some plants overexpressed TPS7_b.

### Example 3

### Cloning of TPS7_a Under the RAB17 Promoter and Transformation

The ZmTPS7_a coding sequence (SEQ ID NO: 19 encoding the protein sequence SEQ ID NO: 7) was codon optimized for maize expression by a gene synthesis service provider and cloned into the pUC57 vector (Genscript). The optimized ZmTPS7_a sequence was linked to the drought inducible Zea mays Rab17 promoter (Vilardell et al., 1991) (SEQ ID NO: 18) and a Ubi4_MAR terminator sequence (SEQ ID NO: 22), by performing a restriction enzyme digestion and ligation in the destination binary plasmid pBIOS03092 forming pBIOS02922, thus leading to the cassette of sequence SEQ ID NO: 24.

pBIOS02922 was transferred into agrobacteria LBA4404 (pSB1) according to Komari et al (1996). Maize cultivar A188 was transformed with these agrobacterial strains essentially as described by Ishida et al (1996).

Analysis of the pRab17-TPS7_a transformed corn plants indicated that some plants overexpressed TPS7_a.

### Example 4

### Corn Field Trials

Field trials show that seed yield and the stability of yield is improved as well as drought tolerance.

Hybrids with a tester line were obtained from T3 plants issued from the TPS7 transgenic maize lines (pRbcs-ZmTPS7_b-Rbcs term, pZmRAB17-ZmTPS7_a-Ubi4_MAR term) chosen according to the previous examples.

The transformant (T0) plant was first crossed with the A188 line thereby producing T1 plants. T1 plants were then self-pollinated twice, producing T3 plants which are homozygous lines containing the transgene. These T3 plants were then crossed with the tester line thereby leading to a hybrid. This hybrid is at a T4 level with regards to the transformation step and is heterozygous for the transgene. These hybrid plants are used in field experiments.

Control hybrids are obtained as follows:

Control Equiv corresponds to a cross between an A188 line (the inbred line used for transformation) and the tester inbred line.

Yield was calculated as follows:

During harvest, grain weight and grain moisture are measured using on-board equipment on the combine harvester.

Grain weight is then normalized to moisture at 15%, using the following formula:

Normalized grain weight=measured grain weight×(100−measured moisture (as a percentage))/85 (which is 100−normalized moisture at 15%).

As an example, if the measured grain moisture is 25%, the normalized grain weight will be:

normalized grain weight=measured grain weight×75/85.

Yield is then expressed in a conventional unit (such as quintal per hectare).

Experimental Design:

Field trials are on 3 different locations.

The experimental block comprises 4 replicates. The experimental design was Randomized Lattice blocks in drought stressed locations. Each replicate comprised of two row plots with about up to 70 plants per plot at a density of 75 000 plants/ha.

Controls were used present in this experiment as described above a control equivalent (A188 crossed with the tester line).

A drought stressed location is a location where the grain yield potential of the site has not been reached due to a drought stress.

A non-stressed location is a location where the grain yield potential has been reached by a commercial hybrid variety.

The drought stress intensity is evaluated by measuring the yield lost between the drought stress treatment (WUE) and a reference treatment irrigated with an optimal amount of water, which is at least, equivalent to the maximum evapotranspiration (ETM) of the crop.

A yield loss of −30% is targeted with a common distribution of the drought location between −10% and −40% of yield.

A low drought stressed location is typically a location with a yield lost between 0% and up to −20%, a moderate stressed location between −20% and up to 30%.

The targeted growth stage period is typically from tasseling to R2 growth stage. In a common drought location, the drought stress period can spread out from a period between V10 and R4 growth stage.

### REFERENCES

Anderson, O. D. & Greene, F. C. (1989). TAG Theor. Appl. Genet. Theor. Angew. Genet. 77, 689-700.
Crétin, C., Santi, S., Keryer, E., Lepiniec, L., Tagu, D., Vidal, J. & Gadal, P. (1991). Gene. 99, 87-94.
Delorge, I., Figueroa, C. M., Feil, R., Lunn, J. E. & Van Dijck, P. (2015). Biochem. J. 466, 283-290.
Han, B., Fu, L., Zhang, D., He, X., Chen, Q., Peng, M. & Zhang, J. (2016). Int. J. Mol. Sci. 17, 1077.
Henry, C., Bledsoe, S. W., Siekman, A., Kollman, A., Waters, B. M., Feil, R., Stitt, M. & Lagrimini, L. M. (2014). J. Exp. Bot. 65, 5959-5973.
Ilhan, S., Ozdemir, F. & Bor, M. (2015). Plant Biol. 17, 402-407.
Ishida, Y., Saito, H., Ohta, S., Hiei, Y., Komari, T. & Kumashiro, T. (1996). Nat. Biotechnol. 14, 745-750.
Jiang, W., Fu, F.-L., Zhang, S.-Z., Wu, L. & Li, W.-C. (2010). J. Plant Biol. 53, 134-141.
Komari, T., Hiei, Y., Saito, Y., Murai, N. & Kumashiro, T. (1996). Plant J. Cell Mol. Biol. 10, 165-174.
Kosmas, S. A., Argyrokastritis, A., Loukas, M. G., Eliopoulos, E., Tsakas, S. & Kaltsikes, P. J. (2006). Planta. 223, 329-339.
Leyman, B., Van Dijck, P. & Thevelein, J. M. (2001). Trends Plant Sci. 6, 510-513.
Li, H.-W., Zang, B.-S., Deng, X.-W. & Wang, X.-P. (2011). Planta. 234, 1007-1018.
Lunn, J. E. (2007). Funct. Plant Biol. 34, 550.
Lunn, J. E., Delorge, I., Figueroa, C. M., Van Dijck, P. & Stitt, M. (2014). Plant J. 79, 544-567.
Matsuoka, M., Kyozuka, J., Shimamoto, K. & Kano-Murakami, Y. (1994). Plant J. Cell Mol. Biol. 6, 311-319.
Matsuoka, M. & Sanada, Y. (1991). Mol. Gen. Genet. MGG. 225, 411-419.
Needleman, S. B. & Wunsch, C. D. (1970). J. Mol. Biol. 48, 443-453.
Nuccio, M. L., Wu, J., Mowers, R., Zhou, H.-P., Meghji, M., Primavesi, L. F., Paul, M. J., Chen, X., Gao, Y., Haque, E., Basu, S. S. & Lagrimini, L. M. (2015). Nat. Biotechnol. 33, 862-869.
Rathod, J. P., Prakash, G., Vira, C. & Lali, A. M. (2016). Prep. Biochem. Biotechnol. 46, 803-809.
Robert, L. S., Thompson, R. D. & Flavell, R. B. (1989). Plant Cell. 1, 569-578.
Sugiyama, N., Izawa, T., Oikawa, T. & Shimamoto, K. (2001). Plant J. Cell Mol. Biol. 26, 607-615.
Vandesteene, L., Ramon, M., Le Roy, K., Van Dijck, P. & Rolland, F. (2010). Mol. Plant. 3, 406-419.
Vilardell, J., Mundy, J., Stilling, B., Leroux, B., Pla, M., Freyssinet, G. & Pag?s, M. (1991). Plant Mol. Biol. 17, 985-993.
Wilson, R. A., Jenkinson, J. M., Gibson, R. P., Littlechild, J. A., Wang, Z.-Y. & Talbot, N. J. (2007). EMBO J. 26, 3673-3685.
Xie, D. W., Wang, X. N., Fu, L. S., Sun, J., Zheng, W. & Li, Z. F. (2015). J. Genet. 94, 55-65.
Yang, H.-L., Liu, Y.-J., Wang, C.-L. & Zeng, Q.-Y. (2012). PloS One. 7, e42438.
Zang, B., Li, H., Li, W., Deng, X. W. & Wang, X. (2011). Plant Mol. Biol. 76, 507-522.

1. A method for improving yield in plants, said method comprising overexpressing a class II threhalose-6 phosphate synthase (TPS) protein comprising the six following domains:
Domain 1 as set forth in SEQ ID NO: 1: FCKQX₁LWPLFHYMLPX₂CX₃DKX₄ELFDRX₅LFX₆AYVRAN, wherein
X₁ can be Q or H
X₂ can be I or V
X₃ can be L or H
X₄ can be G or D
X₅ can be S or N or T
X₆ can be Q or R
Domain 2 as set forth in SEQ ID NO: 2: DDDX₇VWVHDYHLMLX₈PTX₉LRKX₁₀LHRIKX₁₁GFFLHSPFPSSEIYX₁₂X₁₃LPVRDEILKS LLNADLIGFQTFDYARHFLSCCSRLLGLX₁₄YESKRGX₁₅IGIX₁₆YFGRTVX₁₇LKIL, wherein
X₇ can be F or C or H or Y
X₈ can be L or I or V
X₉ can be F or L
X₁₀ can be R or F
X₁₁ can be I or V or L
X₁₂ can be R or K
X₁₃ can be T or S
X₁₄ can be H or N
X₁₅ can be Y or H
X₁₆ can be E or D
X₁₇ can be S or N
Domain 3 as set forth in SEQ ID NO: 3: LGVDDMDIFKGISLKX₁₈LX₁₉LEX₂₀LLX₂₁RX₂₂PKLRX₂₃KVVLVQIX₂₄NPARSX₂₅GKD, wherein
X₁₈ can be F or L
X₁₉ can be G or A
X₂₀ can be L or F
X₂₁ can be D or E
X₂₂ can be N or T
X₂₃ can be E or G or Q
X₂₄ can be I or V
X₂₅ can be T or I or P
Domain 4 as set forth in SEQ ID NO: 4: AASDCCIVNAX₂₆RDGMNLX₂₇PYEYTVCRQGN, wherein
X₂₆ can be V or L
X₂₇ can be V or I
Domain 5 as set forth in SEQ ID NO: 5: HTSTLIVSEFVGCSPSLSGAFRVNPWSX₂₈X₂₉DVADAL, wherein
X₂₈ can be V or M or I
X₂₉ can be D or E
Domain 6 as set forth in SEQ ID NO: 6: RCWX₃₀X₃₁GFGLNFRX₃₂IALSPGFRX₃₃LSX₃₄EH, wherein
X₃₀ can be A, T
X₃₁ can be I, T
X₃₂ can be V, I
X₃₃ can be K, R
X₃₄ can be S, L;
said protein having at least 70% sequence identity with SEQ ID NO: 7.

2. The method according to claim 1 comprising overexpression of a protein having at least 92% sequence identity with SEQ ID NO: 7.

3. The method according to claims 1 or 2 wherein the protein is of sequence SEQ ID NO: 8.

4. The method according to claims 1 or 2 wherein the protein is of sequence SEQ ID NO: 7.

5. The method according to anyone of claims 1 or 4 wherein overexpression is carried out by transforming the plant with a vector comprising a promoter functional in plants and a nucleic acid sequence encoding the protein as defined in anyone of claims 1 to 4.

6. The method according to claim 5 wherein the promoter functional in plants is active in leaf tissues.

7. The method according to claim 6 wherein the promoter functional in plants is selected among a group consisting of a rbcs promoter and a rab17 promoter.

8. The method according to anyone of claim 1 to 7 wherein the yield in plants is improved under drought conditions.

9. A method to identify a plant with improved yield comprising the step of identifying in a population of plants, the plants overexpressing the class II TPS protein as defined in anyone of claims 1 to 7.

10. A method of growing plants comprising the steps of:
(i) sowing plant seeds, wherein said plant seeds originate from plants overexpressing the class II TPS protein as defined in anyone of claims 1 to 7, and
(ii) growing plants from these sowed seeds.

11. A method of growing plants according to claim 10, wherein the growing phase (ii) is made under drought stress.

12. A nucleic acid construct comprising a rab17 promoter operably linked to a nucleic acid sequence encoding the class II TPS protein as defined in claims 1 or 2.

13. The nucleic acid according to claim 12 wherein the nucleic acid sequence encodes a protein of SEQ ID NO: 7.

14. The nucleic acid according to claim 12 wherein the nucleic acid sequence encodes a protein of SEQ ID NO: 8.

15. A transgenic plant comprising the nucleic acid construct of anyone of claims 12 to 14.


SEQUENCE LISTING (24 sequences)

SEQ ID NO: 1 - protein, 36 aa, Artificial Sequence
  Domain 1
  misc_feature (5)..(5): X may be Q or H
  misc_feature (16)..(16): X may be I or V
  misc_feature (18)..(18): X may be L or H
  misc_feature (21)..(21): X may be G or D
  misc_feature (27)..(27): X may be S or N or T
  misc_feature (30)..(30): X may be Q or R
FCKQXLWPLFHYMLPXCXDKXELFDRXLFXAYVRAN

SEQ ID NO: 2 - protein, 105 aa, Artificial Sequence
  Domain 2
  VARIANT (4)..(4): X may be F or C or H or Y
  VARIANT (15)..(15): X may be L or I or V
  VARIANT (18)..(18): X may be F or L
  VARIANT (22)..(22): X may be R or F
  VARIANT (28)..(28): X may be I or V or L
  VARIANT (43)..(43): X may be R or K
  VARIANT (44)..(44): X may be T or S
  VARIANT (83)..(83): X may be H or N
  VARIANT (90)..(90): X may be Y or H
  VARIANT (94)..(94): X may be E or D
  VARIANT (101)..(101): X may be S or N
DDDXVWVHDYHLMLXPTXLRKXLHRIKXGFFLHSPFPSSEIYXXLPVRDEILKSLLNADLIGFQTFDYARHFLSCCSRLLGLXYESKRGXIGIXYFGRTVXLKIL

SEQ ID NO: 3 - protein, 48 aa, Artificial Sequence
  Domain 3
  VARIANT (16)..(16): X may be F or L
  VARIANT (18)..(18): X may be G or A
  VARIANT (21)..(21): X may be L or F
  VARIANT (24)..(24): X may be D or E
  VARIANT (26)..(26): X may be N or T
  VARIANT (31)..(31): X may be E or G or Q
  VARIANT (39)..(39): X may be I or V
  VARIANT (45)..(45): X may be T or I or P
LGVDDMDIFKGISLKXLXLEXLLXRXPKLRXKVVLVQIXNPARSXGKD

SEQ ID NO: 4 - protein, 29 aa, Artificial Sequence
  Domain 4
  VARIANT (11)..(11): X may be V or L
  VARIANT (18)..(18): X may be V or I
AASDCCIVNAXRDGMNLXPYEYTVCRQGN

SEQ ID NO: 5 - protein, 35 aa, Artificial Sequence
  Domain 5
  VARIANT (28)..(28): X may be V or M or I
  VARIANT (29)..(29): X may be D or E
HTSTLIVSEFVGCSPSLSGAFRVNPWSXXDVADAL

SEQ ID NO: 6 - protein, 27 aa, Artificial Sequence
  Domain 6
  VARIANT (4)..(4): X may be A or T
  VARIANT (5)..(5): X may be I or T
  VARIANT (13)..(13): X may be V or I
  VARIANT (22)..(22): X may be K or R
  VARIANT (25)..(25): X may be S or L
RCWXXGFGLNFRXIALSPGFRXLSXEH

SEQ ID NO: 7 - protein, 865 aa, Zea mays
MVSKSYSNLLDLTSGDGFDFRQPFKSLPRVVTSPGIISDTDWDTISDGDSVGSASSTERKIIVANFLPLNCTRDETGVLSFSLDHDALLMQLKDSFSNETDVVYVGSLKVQVDPGEQDQVAQKLLREYRCIPTFLPSDLQQQFYHGFCKQQLWPLFHYMLPICLDKGELFDRSLFQAYVRANKLFADKVMEAINADDDFVWVHDYHLMLLPTFLRKRLHRIKIGFFLHSPFPSSEIYRTLPVRDEILKSLLNADLIGFQTFDYARHFLSCCSRLLGLHYESKRGYIGIEYFGRTVSLKILSVGVHIGRLESVLKLPATVSKVQEIEQRYKGKILMLGVDDMDIFKGISLKFLGLELLLDRNPKLREKVVLVQIINPARSTGKDVQEAITEAVSVAERINTNYGSSSYKPVVLIDHHIPFYEKIAFYAASDCCIVNAVRDGMNLVPYEYTVCRQGNEEIDKLRGLGKDTHHTSTLIVSEFVGCSPSLSGAFRVNPWSVDDVADALCRATDLTESEKRLRHEKHYRYVSTHDVAYWARSFAQDLERACKDHYSRRCWAIGFGLNFRVIALSPGFRKLSSEHFVSSYNKASRRAIFLDYDGTLVPQSSINKAPSEEVISVLNTLCNDPKNIVFIVSGRGRDSLDEWFSPCEKLGLAAEHGYFIRWSKEAAWESSYSRPQQEWKHIAEPVMQVYTETTDGSSIESKESALVWHYLDADHDFGSFQAKELQGHLERVLSNEPVVVKCGHYIVEVKPQGVSKGLAVNKLIHTLVKNGKAPDFLMCVGNDRSDEDMFESINGMTSNAVLSPTMPELFACSVGQKPSKAKYYVDDTSEVIRLLKNVTRIPSQRQDVSASHGRVTFRGVLDYVD

SEQ ID NO: 8 - protein, 868 aa, Zea mays
MVSKSYSNLLDMTPGDGFDFRRPFKSLPRVVTSPSIISDHDWDSISDGDSVGSAFSIERKIIVANFLPLNCTRDETGELSFSLDHDSLLMQLKDGFSNETDAVYVGSLKVHVDPREQDQVAQKLLREYRCIPTFLPSDLQQQFYHGFCKQQLWPLFHYMLPICLDKGELFDRTLFQAYVRANKLFADKVMEAINTDDDYVWVHDYHLMLLPTFLRKRLHRIKIGFFLHSPFPSSEIYRTLPVRDEILKSLLNADLIGFQTFDYARHFLSCCSRLLGLHYESKRGYIGIEYFGRTVSLKILSVGVHVGRLESVLKLPATVSKVEEIEQRYKGKILMLGVDDMDIFKGISLKLLALELLLDRNPKLREKVVLVQIINPARSTGKDVQEAITEAVSVAERVNTKYGSSSYKPVVLIDNRIPFYEKVAFYAASDCCIVNAVRDGMNLVPYEYTVCRQGNEEIDRVRGLDKDTHHTSTLIVSEFVGCSPSLSGAFRVNPWSVDDVADALCRATDLSESEKRLRHEKHYRYVSTHDVAYWAHSFAQDLERACRDHYSRRCWAIGFGLNFRVIALSPGFRKLSSEHFVSSYNRASRRAIFLDYDGTLVPQSSINKAPSEEVISILNTLCNDPKNVVFIVSGRGRDSLDEWFSPCEKLRLAAEHGYFIRWSKEAAWESSYSSPRQEWKHIAEPVMQVYTETTDGSSVESKESALVWHYLDADHDFGSFQAKELKDHLERVLSNEPVVVKCGHYIVEVKPQGVSKGRAVDKLIQALANNNGKAQDFLMCVGNDRSDEDMFECINGMASNDVSSTTVPEVFACSVGQKPSKAKYYVDDTSEVIRLLRDATRFSSSQRREDVNASRGRVTFRDALDYVD

SEQ ID NO: 9 - protein, 865 aa, Sorghum bicolor
MVSKSYSNLLEMTSGDGFDFRQPFKSLPRVVTSPGIISDPDWDTISDGDSVGSASSTERKIIVANFLPLNCTRDDTGKLSFSLDHDALLMQLKDGFSNETDAVYVGSLKVQVDPSEQDQVAQKLLREYRCIPTFLPSDLQQQFYHGFCKQQLWPLFHYMLPICLDKGELFDRNLFQAYVRANKLFADKVMEAINTDDDCVWVHDYHLMLLPTFLRKRLHRIKIGFFLHSPFPSSEIYRTLPVRDEILKSLLNADLIGFQTFDYARHFLSCCSRLLGLHYESKRGYIGIEYFGRTVSLKILSVGVHVGRLESVLKLPATISKVQEIEQRYKGKILMLGVDDMDIFKGISLKLLGLELLLDRNPKLREKVVLVQIINPARSTGKDVQEAITEAVSVAKRINTKYGSSSYKPVVLIDHRIPFYEKIAFYAASDCCIVNAVRDGMNLVPYEYTVCRQGNEEIDKLRGLDKDTHHTSTLIVSEFVGCSPSLSGAFRVNPWSVDDVADALCRATDLTESEKRLRHEKHYRYVSTHDVAYWARSFAQDLERACKDHYSRRCWAIGFGLNFRVIALSPGFRKLSLEHFVSSYNKASRRAIFLDYDGTLVAQSSINKAPSEEVISILNTLCNDPKNVVFIVSGRGRDSLDEWFSPCEKLGVAAEHGYFIRWSKEAAWESSYSSPQQEWKHIAEPIMQVYTETTDGSSIESKESALVWHYLDADHDFGSFQAKELQDHLERVLSNEPVVVKCGHYIVEVKPQGVSKGLAVNKLIHTLVKNGKAPDFLMCVGNDRSDEDMFECINGMTSNDAISPTAPEVFACSVGQKPSKAKYYVDDTSEVIRLLKNVTRVSSQREDVNASHGRVTFRDVLDYVD

SEQ ID NO: 10 - protein, 861 aa, Sorghum bicolor
MVLKSYTNLLDMCCEDVFQQPLRSLPHVVTSPGIISDPDCESSNDGNLVGSTHICFKRKIIVANFLPMICAKNEATGEWSFAMDDNQLLVQLKDGFPIDNEVIYVGSLNVQVDPSEQDRVSQKLFKEHRCIPTFLPADLQQQFYHIFCKQHLWPLFHYMLPVCHDKGELFDRSLFQAYVRANKIFADKVVEAVNSDDDCVWVHDYHLMLIPTFLRKKLHRIKVGFFLHSPFPSSEIYRTLPVRDEILKSLLNADLIGFQTFDYARHFLSCCSRLLGLNYESKRGHIGIEYFGRTVSLKILAAGVHVGRLESMLKLPATINKVQEIESRYSGKLVILGVDDMDIFKGISLKLLGLELLLERTPKLRGKVVLVQIVNPARSIGKDVEEAKYEAVSVAQRINDKYGSANYKPVVLIDYSIPFYEKIAFYAASDCCIVNAVRDGMNLIPYEYTVCRQGNEDIDKLRGVNKSSSHTSTLIVSEFVGCSPSLSGAFRVNPWSMEDVADALYNATDLTQYEKNLRHEKHYRYVRSHDVAYWAHSFDQDLERACREQYSQRCWTTGFGLNFRIIALSPGFRRLSLEHLTSSYKKANRRMIFLDYDGTLVPQASHDKSPSAELISILNSLCNDMKNTVFIVSGRGRDSLSEWFVSCENLGIAAEHGYFIRWNKAAEWETSLSGLHSEWKLIVEPIMHLYMETTDGSFIEQKESALVWHYQNTDHDFGLCQAKELVGHLERVLSNEPVAVRRGHQIVEVKPQGVNKGISVDKIIQTMVSKGDVPDLLMCIGNDRSDEDMFESINKATSLSEPAIPEVFACSVGPKASKANYYVDGCSEVIRLLKGVTAISPQKDTVSHSHAVFKDTLEVIS

SEQ ID NO: 11 - protein, 864 aa, Zea mays
MVLKSHTNLLDMCCEDVFDFQQPLRSPRHVVNSPGIISDPDWESSNDGNSVGSMPFCFKRKIIVANFLPVICAKNEATGEWSFAMDDNQLLVQLKDGFPIGNEVIYVGSLNVQVDPIEQDRVSQKLFKEHRCVPTFLPAELQHQFYHIFCKQHLWPLFHYMLPVCHDKDELFDRSLFQAYVRANKIFADKIVEAVNSDDDCVWVHDYHLMLIPTLLRKKLHRIKVGFFLHSPFPSSEIYRTLPVRDEILKSLLNADLIGFQTFDYARHFLSCCSRLLGLNYESKRGHIGIEYFGRTVSLKILAAGVHVGRLEATLRLPATIKKVQEIESRYSGKLVILGVDDMDIFKGISLKLLGLELLLERTPKLRGKVVLVQIVNPARSIGKDIEEAKYEAESVAQRINDKYGSANYKPVVLIDYSIPFYEKIAFYAASDCCIVNAVRDGMNLIPYEYTVCRQGNEELDKLRGLNKSSSHTSTLIVSEFVGCSPSLSGAFRVNPWSMEDVADALYSVTDLTRYEKNLRHEKHYRYVRSHDVAYWARSFDQDLDKACIEQYSQRCWTTGFGLNFRVIALSPGFRRLSLEHLASSYKKANRRMIFLDYDGTLVPQTSHDKSPSAELISTLNSLCSDMKNTVFIVSGRGRDSLSEWFASCENLGIAAEHGYFIRWNKAAEWETSFSGIYSEWKLIADPIMHVYMETTDGSFIEPKESALVWHYQNTDHDFGSCQAKELVSHLERVLSNEPVVVRRGHQIVEVKPQGVSKGISVDKIIRTLVSKGEVPDLLMCIGNDRSDEDMFESINRATSLSELPAAPEVFACSVGPKASKANYYVDGCDEVIRLLKGVTAVSLQKDTAGHSHAAFEDTLEVVS

SEQ ID NO: 12 - protein, 862 aa, Brachipodium distachyon
MVSKSYSNLLEMSCGDSVDFRQPFKSLPRVVTSPGLISDPDWDSRSDDDSVGSASFTERKIIVANFLPLNCMKDEAGQWSFSKDDDALLMQLKDGFSDETDVIYVGSLKVQIDPSDQDHVAQKLLREYRCIPTFLPSELQQQFYHGFCKQQLWPLFHYMLPICLDKGELFDRSLFRAYVRANKIFADKVMEAINTDDDCVWVHDYHLMLLPTFLRKRLHRIKLGFFLHSPFPSSEIYRTLPVRDEILKSLLNADLIGFQTFDYARHFLSCCSRLLGLHYESKRGYIGIEYFGRTVSLKILSVGVHVGRLESILKLPSTASKVQEIEQRYKGKMLMLGVDDMDIFKGISLKLLGLELLLDRNPKLRGKVVLVQIVNPARSPGKDVEEAITEAVSVAERINVKYGSADYKPVVLIDNRIPFSEKIAFYAASDCCIVNAVRDGMNLVPYEYTVCRQGNDVMDKHRGFDKNHHHTSTLIVSEFVGCSPSLSGAFRVNPWSVDDVADALHSATDLTESEKRLRHDKHYRYVSTHDVAYWARSFIQDLERACKDHYSQRCWAIGFGLNFRVIALSPGFRKLSLEHFLSSYNKATRRAIFLDYDGTIVPQSSINKTPSAELISILNSLCNDPKNDVFIVSGRGRNSLDEWFAPCEKLGIAAEHGYFVRWNQATEWESNYSSPDREWKHIAEPVMQVYTETTDGSFIEPKESALVWHYLDADHDFGSCQAKELLDHLERVLSNEPVGVKCGHFIVEVKPQGVSKGLAVDKLIRTLINNGKTPDFLMCIGNDRSDEDMFESINSKACSSAFATIPEVLACSVGQKPSKAKYYVDDTAEVIRLLKNASGVSSQQEVVSQGRVSFRHVLDYVD

SEQ ID NO: 13 - protein, 863 aa, Setaria italica
MVSKSYSNLLDMTSGDGFDFRQPFKSLPRVVTSPGIISDPDWDTRSDDDSVGSASFSERKIIVANFLPLNCTRDEAGQLSFSLDDDALLVQLKHGFSNETDVVYVGSLKIQVDPSEQDQVAQKLLREYRCIPTFLPSDLQQQFYHGFCKQQLWPLFHYMLPICLDKGELFDRSLFQAYVRANKLFADKVMEVINTDDDYVWVHDYHLMLLPTFLRKRLHRIKLGFFLHSPFPSSEIYRTLPVRDEILKSLLNADLIGFQTFDYARHFLSCCSRLLGLHYESKRGYIGIEYFGRTVSLKILSVGVHVGRLESVLNLPATVSKVQEIEQRYKGKMLMLGVDDMDIFKGISLKLLGLELLLERNPKLRQKVVLVQIINPARSTGKDVQEAITETVSVAERINRKYGSSGYNPVVLIDHHIPFYEKIAFYAASDCCIVNAVRDGMNLVPYEYTVCRQGNEEIDKLRGFDKDTSHTSTLIVSEFVGCSPSLSGAFRVNPWSVDDVADALCHATDLTESEKRLRHEKHYRYVSTHDVAYWARSFAQDLERACKDHYSRRCWAIGFGLNFRVIALSPGFRKLSSEHFVSCYNKASRRAIFLDYDGTLVPQSSINKAPSAEVISILKTLCNDPKNNVFIVSGRGRDSLDEWFSPCEKLGIAAEHGYFVRWSKEAEWESSYPRTQREWKHIAEPVMKVYTETTDGSSIEPKESALVWHYLDADHDFGSCQAKELQDHLERVLSNEPVVVKCGHYIVEVKPQGVSKGLAVDKLIRSLVNNGKAPDFLMCIGNDRSDEDMFESINGMTSNTVLSPTVPEVFACSVGQKPSKAKYYVDDTTEVIRLLKNVTRSSSQREDVSHGRVTFRDVIDFVE

SEQ ID NO: 14 - protein, 864 aa, Setaria italica
MVLNSFSNLLDICSEDVFDFQQPLRSLPCAVTSPGIRSDPDWESSNGSNLIGSAPPCLTRKIVVANFLPLNCTKDEATREWSFAVDDNQLLVQLKDGFPIDSEVIYVGSLNVQVDPSEQDQVSQKLFKEHKCIPTFLPADLQQQFYHSFCKQHLWPLFHYMLPVCHDKGELFDRSLFQAYVRANQIFADKVMEAVNSDDDCVWVHDYHLMLVPTFLRKKLHRIKVGFFLHSPFPSSEIYKTLPVRDEILKSLLNADLIGFQTFDYARHFLSCCSRLLGLNYESKRGHIGIEYFGRTVNLKILAAGVHVGRLESMLKLPVTISKVQEIENRYRGKLVILGVDDMDIFKGISLKLLGLELLLERTPKLRGKVVLVQIVNPARSIGKDVEEAKNEAVSVAQRINDKYGSANYKPVVLIDYSIPFYEKIAFYAASDCCIVNAVRDGMNLIPYEYTVCRQGNEDIDKLRGSDKSSLHTSTLIVSEFVGCSPSLSGAFRVNPWSVEDVADALYSATDLTQFEKIQRHEKHYRYVKSHDVTYWARSFDQDLERTCKEQDSRRCWTTGFGLNFRVIALSPGFRRLSLEHFASSYKKANRRVIFLDYDGTLVPQSSLNKAPSAEVISILNSLCNDTKNTVFIVSGRGRNSLSEWFDSCENLGIAAEHGYFIRWNKAAEWETSSSGQCSEWKLIADPVMHVYTETTDGSSIECKESALVWHYQNTDHDFGSCQAKELVSHLERVLANEPVVVKRGHQIVEVKPQGVSKGIAVDKIIRTLVSKGEVADLLMCIGNDRSDEDMFESINKATSLAELPAIPEVFACSVGPKASKANYYVDGCSEVIRLLKGVIDVSSQKDTTSHSHVNSNDILEVVS

SEQ ID NO: 15 - protein, 862 aa, Oryza sativa
MVSKSYSNLLEMSCGDGVDFRQPFKSLPRVVTSPGIISDPDWDTRSDGDSVGSASSVERKIIVANFLPLNCTKDEAGQWSFSRDDDALLMQLKDGFSNETDVIYVGSLKVQVDPSEQDQVAQKLLRDYRCIPTFLPPDLQQQFYHGFCKQQLWPLFHYMLPICLDKGELFDRSLFQAYVRANKLFADKVMEAINTDDDHVWVHDYHLMLLPTFLRKRLHRIKLGFFLHSPFPSSEIYRSLPVRDEILKSLLNADLIGFQTFDYARHFLSCCSRLLGLHYESKRGYIGIDYFGRTVSLKILSVGVHVGRLESILKLPATVKKVQEIEQRYKGKMLMLGVDDMDIFKGISLKLLGLELLLDRNPKLRGKVVLVQIVNPARSTGKDVEEAITESVSVAERINLKYGSVDYKPVVLIDHRIPFYEKIAFYAASDCCIVNALRDGMNLVPYEYTVCRQGNEEIDNARGSDTNCHHTSTLIVSEFVGCSPSLSGAFRVNPWSVDDVADALHHATDLTESEKRLRHEKHYRYVRSHSVAYWAHSFAQDLERACKDHYSRRCWAIGFGLNFRVIALSPGFRKLSLEHFASSYNKATRRAIFLDYDGTLVPQSSINKAPSDELITILNSLCDDPKNDVFIVSGRARSLLDEWFAPCQKLGIAAEHGYFVRWNKAAEWESSYPNHDFEWKHIAEPVMQVYTETTDGSSIEPKESALVWHYLDADHDFGSCQAKELLGHLERVLSNEPVVVKCGHYIVEVKPQGVSKGLVVDKVIHRLMNNGKTPDFVVCIGNDRSDEDMFKSIDSMTSSSAFPAVPEVFACSVGQKPSKAKYYVDDAGEVVRLLKNVAGISSHREAVSHGRVTFRDVMDYVE

SEQ ID NO: 16 - protein, 863 aa, Oryza sativa
MVSKSYTNLLDMSGEDVFDFQQPFRSLPRFVTSPSITSNPDWDTSNADDSVGPASCCVRKIIVSNFLPLNCTKDEATGQWSFSMDDNQLLVQLKDGFPMESEVVYVGSLNAEVDPGEQDQLSQKLFREYKCIPTFLPADLQQQFYHGFCKQQLWPLFHYMLPICLDKGELFDRSLFQAYVRANKIFGDKVMEAINSDDDCVWVHDYHLMLLPTFLRKKLHRIKIGFFLHSPFPSSEIYRTLPVRDEILKSLLNADLIGFQTFDYARHFLSCCSRLLGLNYESKRGHIGIEYFGRTVSLKILAVGVHVGRLESVLRLPATISKVQEIEQRYKGKMVMLGVDDMDIFKGISLKLLGLEFLLERTPKLRGKVVLVQIINPARSTGKDVEEAINEAVSVAERINIKYGSAEYKPVILIDYPIPSYEKIAYYAASDCCIVNAVRDGMNLIPYEYTVCRQGNEEIDKLRGVDKSSHHTSTLIVSEFVGCSPSLSGAFRVNPWSIEDVADALYKAMDLTQSERKLRHDKHYRYVKTHDVAYWARSFSQDLDRACKDHYSRRCWTTGFGLNFRVIALSPGFRRLSLEHFASSYKKTNRRLIFMDYDGTLVPQSSVNKVPSAEVISILTSLCNDPKNCVFIVSGRDRTTLSEWFASCDKLGIAAEHGYFIRWNKEGEWETSSSAQDCEWKNIAEPIMEVYKETTDGSAIETKESGLVWHYQDADHDFGSCQAKELVSHLERVLANEPVVVKRGHQIVEVKPQGVSKGIAVDTVIRTVINNENAPDFLMCIGNDRSDEDMFESINEAVSRSVFPTAPDVFACSVGQKASKAKYYVDGCSEVIRLLKGVTAITPRREVISQSQVTFRDILEVVS

SEQ ID NO: 17 - DNA, 968 bp, Zea mays
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgattcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcag

SEQ ID NO: 18 - DNA, 444 bp, Zea mays
aaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatc

SEQ ID NO: 19 - DNA, 2598 bp, Zea mays
atggtctccaagtcctactctaatctgctcgatctcacctccggcgatgggttcgatttccgccagcccttcaagtccctgccacgcgtggtcacctccccaggcatcattagcgatacagactgggatactatctccgacggcgattcagtggggtccgcttccagcacggagcggaagatcattgtcgccaatttcctgcctctcaactgcacccgcgacgagaccggcgtgctgtcgttctctctcgaccacgatgcgctcctgatgcagctgaaggattcattctccaacgagacagacgttgtctacgtgggcagcctcaaggttcaggtggatccaggggagcaggaccaggtggcccagaagctcctgagggagtaccggtgcatcccgaccttcctgccctcggacctccagcagcagttctaccacggcttctgcaagcagcagctgtggccactcttccattacatgctgcctatctgcctcgataagggcgagctgttcgaccgcagcctgttccaggcctacgtcagggcgaataagctgttcgccgataaggttatggaggctatcaacgccgacgatgacttcgtctgggttcacgactaccatctgatgctcctgccgaccttcctgaggaagcggctccaccgcatcaagattggcttcttcctccattccccgttcccctcgtctgagatctaccgcacactccccgtcagggacgagattctgaagtccctcctgaacgccgatctcatcggcttccagactttcgactacgccaggcacttcctgagctgctgctcgcgcctcctgggcctccattacgagagcaagcggggctacatcgggattgagtacttcgggcgcacagtctcgctgaagatcctctctgtgggcgtccacattgggcggctggagtcggtgctgaagctcccagcgactgtgtctaaggtccaggagatcgagcagaggtacaagggcaagattctgatgctcggggtggatgacatggacatcttcaagggcatttccctcaagttcctgggcctggagctcctcctcgaccggaatccgaagctccgcgagaaggtcgttctggtccagatcattaacccggctaggtctacgggcaaggacgtgcaggaggctattacggaggccgtttcagtggcggagaggatcaacaccaattacggctcatccagctacaagccagtggtcctgatcgaccaccatattcctttctacgagaagattgcgttctacgccgcgtccgattgctgcatcgttaatgctgtgcgcgacggcatgaacctcgtcccgtacgagtacaccgtttgcaggcaggggaatgaggagattgataagctcaggggcctggggaaggacacccaccatacatcaactctgatcgtgtccgagttcgtgggctgctccccctccctctctggggcgttcagggtcaacccctggtccgtcgatgacgttgcggatgccctctgcagggctacggacctgacggagtccgagaagcgcctgaggcacgagaagcattaccgctacgtgtccacgcacgacgtggcgtactgggctaggtccttcgcgcaggacctggagcgggcttgcaaggaccattacagccgcaggtgctgggctatcggcttcgggctcaacttccgcgtcattgccctgtcgcccggcttcaggaagctctcgtctgagcacttcgtgtcatcctacaataaggcctcccgccgcgccatcttcctcgactacgatggcaccctggtgccgcagagctcgatcaacaaggcccccagcgaggaggtcatctcggtcctgaacacgctctgcaatgacccgaagaacatcgttttcattgtgtccggccgggggcgcgatagcctggacgagtggttctccccgtgcgagaagctgggcctcgctgctgagcacgggtacttcatccgctggagcaaggaggcggcttgggagtcttcatactcccgcccgcagcaggagtggaagcacatcgcggagcctgtcatgcaggtttacacggagaccacggacggctccagcatcgagtctaaggagtcagccctcgtctggcactacctggacgcggatcatgacttcggctcgttccaggctaaggagctccaggggcacctggagcgcgtgctctctaatgagccagttgtggtcaagtgcggccattacatcgtcgaggttaagccgcagggcgtgagcaaggggctggcggtcaataagctcatccacaccctggttaagaacggcaaggccccggatttcctcatgtgcgtggggaacgaccgctccgatgaggacatgttcgagagcatcaatggcatgacatctaacgccgtcctgtcaccaactatgcctgagctgttcgcctgctctgtgggccagaagccatcaaaggctaagtactacgttgatgacacatccgaggtcatccgcctgctcaagaacgtgactaggattcctagccagcggcaggatgtgtcggcgagccacgggcgggtcactttccggggcgttctggactacgttgattag

SEQ ID NO: 20 - DNA, 2607 bp, Zea mays
atggttagcaagtcctacagcaacctcctcgacatgacacctggggatggcttcgatttccgccgcccgttcaagtctctgcctcgcgtggtcacatcgccgtctatcattagcgatcacgactgggattcaatttccgacggcgattctgttgggtcagcgttctccatcgagcgcaagatcattgtcgctaacttcctgcccctcaattgcacaagggacgagactggcgagctgagcttctcgctcgaccacgattctctcctgatgcagctgaaggacggcttctcaaacgagaccgatgcggtttacgtggggtccctcaaggtccacgttgacccccgcgagcaggatcaggtggctcagaagctcctgagggagtaccggtgcatcccaacgttcctgccttccgacctccagcagcagttctaccacggcttctgcaagcagcagctgtggccgctgttccattacatgctgcccatctgcctcgacaagggggagctgttcgatcggaccctgttccaggcctacgtgcgcgctaacaagctgttcgcggacaaggtcatggaggctatcaataccgacgatgactacgtgtgggtccacgattaccatctgatgctcctgccaacgttcctgcgcaagaggctccacaggatcaagattggcttcttcctccattcgccgttcccctccagcgagatctacaggacactccctgtgcgggacgagattctgaagtccctcctgaacgcggacctgatcggcttccagactttcgattacgctaggcacttcctgagctgctgctcgcggctcctgggcctccattacgagtcgaagcggggctacatcgggattgagtacttcgggcgcacagtgtccctgaagatcctcagcgttggcgtgcacgtcgggcgcctggagtccgtgctcaagctcccggccactgtgtcgaaggtcgaggagatcgagcagaggtacaagggcaagattctgatgctcggggtggatgacatggacatcttcaagggcatttccctcaagctcctggcgctggagctcctgctcgatcgcaacccgaagctcagggagaaggttgtgctggtgcagatcattaatccagctaggtccaccggcaaggacgtccaggaggctatcacggaggccgttagcgtggcggagagggtgaacaccaagtacgggtcgtcttcatacaagccggtcgttctgatcgacaatcggattcccttctacgagaaggtcgctttctacgccgcgtccgactgctgcatcgttaacgccgtgcgggatggcatgaatctcgttccatacgagtacacagtgtgccgccagggcaacgaggagattgaccgggtgcgcgggctggacaaggatactcaccatacctcgacgctcatcgtctctgagttcgttggctgctctccatcactgtccggggcgttcagggtcaatccttggagcgtcgatgacgttgctgacgctctctgcagggctaccgatctgagcgagtcggagaagaggctgcggcacgagaagcattaccgctacgtgagcacgcacgacgtcgcgtactgggctcattcgttcgctcaggacctggagagggcttgcagggatcactactcccgcaggtgctgggctatcggcttcgggctcaacttccgcgtgattgcgctgagcccgggcttcaggaagctctccagcgagcacttcgtctcgtcttacaacagggcctcccgccgcgccattttcctcgactacgatggcaccctggtgccacagtcatccatcaataaggccccttcagaggaggtcatctccattctgaatacgctctgcaacgacccgaagaatgtggtcttcatcgtgtccggccgcggcagggactccctcgacgagtggttctctccctgcgagaagctgaggctcgctgccgagcacggctacttcattcggtggtccaaggaggcggcttgggagagctcgtactcttcaccgcgccaggagtggaagcatatcgcggagcccgtgatgcaggtctacacggagaccacggacggctccagcgtcgagtctaaggagtcagctctcgtttggcactacctggacgccgatcatgacttcggctcgttccaggccaaggagctgaaggaccacctggagcgcgtgctctctaacgagccagttgtggtcaagtgcggccattacattgtcgaggttaagcctcagggcgtctccaagggcagggccgttgacaagctcatccaggctctggccaacaataacggcaaggctcaggacttcctgatgtgcgtcgggaacgataggtctgatgaggacatgttcgagtgcatcaacggcatggcgtcaaatgacgtgtcgtctacaactgttccagaggtgttcgcctgctcagtcgggcagaagccttccaaggcgaagtactacgtggatgacacatccgaggtcatccggctgctcagggacgctactcggttctcatccagccagaggcgggaggatgttaacgcctcccgcggcagggtgaccttccgcgatgccctcgactacgtggattga

SEQ ID NO: 21 - DNA, 537 bp, Zea mays
tagaccgcgcccgccggccgccccccgccggctagctagctagctagctagctcctgcgtgagctagtagctagtgccatgcgtcgtctctgtcgttcggttttgcttcgggtcaccgtaccctttgcttgcttggtttcttctttccttttttccttttttttttcttcttttccccggccatggttcctttgctttcagcagttctctgctggatgtgatgtatccattgttgcaagcatggccttgcattggctacctctatacctgctacaaactactgcaacgcctatatatacttggggtgaggaacatgtgaatgcaagctccggctatcatatacatgtaatatggatacaaactatatatataaatccgccgaggcgccgactaatactatacgacgacaccgtgttaagttaatatataactggtgctttttatttatatatctgtctcatcatatatatatgctaattaatggatgtgtgtcctcttcacttcaattccttctttcctttcctatgctttgagatc

SEQ ID NO: 22 - DNA, 1661 bp, Saccharum officinarum
tcctgggccatgaagctgtccttccaggttcacaagtctggtgccttcttctgtccctccgatggagattatctgcatgtcgtggtcgtgtcctgatcgaatcctcgttgaatccctatgtttttcttcaagaaatgtgagtcctatgtcagtctggttgcgtttgtgaacatttctgctgctgagcagcactttggctggaactgtgcaatgaaataaatggaaccctggtttctggttatgtgtgtgttagctaatgtttttgaagtggaagctctaatcttctatcgcgttgctactacaattctgcttgtgttttgatgttcttggtttctgttagttggttcagaggaagttttgcttccacagactaagatgcagttgaactttggttgccctggtttctagatttcatttgtgctggttgagtgatagtaagaaacaaccggtgttcacatataatcaggttttgtgctgctcgagtgatcgtcaaaaaccaccggtgttcacatctaaaaaggtttcgatccccaggtttagatctcccgtttaattccaaaaaaaaagttctgtgtacttgcatttagttgggtggttgatgctggaaagagtaactttcaagagtaataatctttggtgactactctgtttcaactgatcaatccctaggaaaggtacacctttacttagggaagaaattcttagaaccttgcactttgtttcaactgataatagtatactttattagataaaaaatattcagatatattagacaccggatgtcatccactcatccttacaaacctctgtcatggtcctgcagaaatgtttgccagctccagtggcttcctgataaatctgtggagtgcctgttaatcggctgccaatttttgctgagcactgtatatatgttagtaagtactattgggccaccaattcgattttgacacagcactattggtccaccaattcgattctgacacagcactgcataatttgaaacgtgttgctccattttgcaaggctacaaatttagatcatgtttagcattctgtgggatacaatatatggatatcgaacaaacttggtatgtcagagaaaaaatagtttattttcaaaactaacatttttaaagccttctatgaactttaaaccttcagcatttgggatcaagatgagtgctcgaacaagagtgcactttttctccaaaataatctactacagagttcttttttatatataaaaaaacttatacttaacagataaatcagactttttctgctccatatcaccttgacaaatcaaagaagcagcaccagcgaagggtattattattgaggtaaatataagatctcgtttactgaaaaagaccgcgtgtttacctaaactaccattttgctttgatagcagcatacatgtgatagaattgcggatcctaccgtgctgactgtgaaggtggtaggggtgagagattggtgggcgaggtctgaacgagcgagaacagtactgcatttactgttcacaaggaggcggcttaggttttgggtctcccagctctctaagggaagctgagaattatgattctcttgcttaattatttcttaaccaaagttataaatatatagcctatgagatcctaatttatggaaataactaaacta

SEQ ID NO: 23 - DNA, 4123 bp, Artificial Sequence
  proZmrbcS_Mut_al - synZmTPS7b - terZmrbcS
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgattcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagtctccaccatggttagcaagtcctacagcaacctcctcgacatgacacctggggatggcttcgatttccgccgcccgttcaagtctctgcctcgcgtggtcacatcgccgtctatcattagcgatcacgactgggattcaatttccgacggcgattctgttgggtcagcgttctccatcgagcgcaagatcattgtcgctaacttcctgcccctcaattgcacaagggacgagactggcgagctgagcttctcgctcgaccacgattctctcctgatgcagctgaaggacggcttctcaaacgagaccgatgcggtttacgtggggtccctcaaggtccacgttgacccccgcgagcaggatcaggtggctcagaagctcctgagggagtaccggtgcatcccaacgttcctgccttccgacctccagcagcagttctaccacggcttctgcaagcagcagctgtggccgctgttccattacatgctgcccatctgcctcgacaagggggagctgttcgatcggaccctgttccaggcctacgtgcgcgctaacaagctgttcgcggacaaggtcatggaggctatcaataccgacgatgactacgtgtgggtccacgattaccatctgatgctcctgccaacgttcctgcgcaagaggctccacaggatcaagattggcttcttcctccattcgccgttcccctccagcgagatctacaggacactccctgtgcgggacgagattctgaagtccctcctgaacgcggacctgatcggcttccagactttcgattacgctaggcacttcctgagctgctgctcgcggctcctgggcctccattacgagtcgaagcggggctacatcgggattgagtacttcgggcgcacagtgtccctgaagatcctcagcgttggcgtgcacgtcgggcgcctggagtccgtgctcaagctcccggccactgtgtcgaaggtcgaggagatcgagcagaggtacaagggcaagattctgatgctcggggtggatgacatggacatcttcaagggcatttccctcaagctcctggcgctggagctcctgctcgatcgcaacccgaagctcagggagaaggttgtgctggtgcagatcattaatccagctaggtccaccggcaaggacgtccaggaggctatcacggaggccgttagcgtggcggagagggtgaacaccaagtacgggtcgtcttcatacaagccggtcgttctgatcgacaatcggattcccttctacgagaaggtcgctttctacgccgcgtccgactgctgcatcgttaacgccgtgcgggatggcatgaatctcgttccatacgagtacacagtgtgccgccagggcaacgaggagattgaccgggtgcgcgggctggacaaggatactcaccatacctcgacgctcatcgtctctgagttcgttggctgctctccatcactgtccggggcgttcagggtcaatccttggagcgtcgatgacgttgctgacgctctctgcagggctaccgatctgagcgagtcggagaagaggctgcggcacgagaagcattaccgctacgtgagcacgcacgacgtcgcgtactgggctcattcgttcgctcaggacctggagagggcttgcagggatcactactcccgcaggtgctgggctatcggcttcgggctcaacttccgcgtgattgcgctgagcccgggcttcaggaagctctccagcgagcacttcgtctcgtcttacaacagggcctcccgccgcgccattttcctcgactacgatggcaccctggtgccacagtcatccatcaataaggccccttcagaggaggtcatctccattctgaatacgctctgcaacgacccgaagaatgtggtcttcatcgtgtccggccgcggcagggactccctcgacgagtggttctctccctgcgagaagctgaggctcgctgccgagcacggctacttcattcggtggtccaaggaggcggcttgggagagctcgtactcttcaccgcgccaggagtggaagcatatcgcggagcccgtgatgcaggtctacacggagaccacggacggctccagcgtcgagtctaaggagtcagctctcgtttggcactacctggacgccgatcatgacttcggctcgttccaggccaaggagctgaaggaccacctggagcgcgtgctctctaacgagccagttgtggtcaagtgcggccattacattgtcgaggttaagcctcagggcgtctccaagggcagggccgttgacaagctcatccaggctctggccaacaataacggcaaggctcaggacttcctgatgtgcgtcgggaacgataggtctgatgaggacatgttcgagtgcatcaacggcatggcgtcaaatgacgtgtcgtctacaactgttccagaggtgttcgcctgctcagtcgggcagaagccttccaaggcgaagtactacgtggatgacacatccgaggtcatccggctgctcagggacgctactcggttctcatccagccagaggcgggaggatgttaacgcctcccgcggcagggtgaccttccgcgatgccctcgactacgtggattgaaactagaccgcgcccgccggccgccccccgccggctagctagctagctagctagctcctgcgtgagctagtagctagtgccatgcgtcgtctctgtcgttcggttttgcttcgggtcaccgtaccctttgcttgcttggtttcttctttccttttttccttttttttttcttcttttccccggccatggttcctttgctttcagcagttctctgctggatgtgatgtatccattgttgcaagcatggccttgcattggctacctctatacctgctacaaactactgcaacgcctatatatacttggggtgaggaacatgtgaatgcaagctccggctatcatatacatgtaatatggatacaaactatatatataaatccgccgaggcgccgactaatactatacgacgacaccgtgttaagttaatatataactggtgctttttatttatatatctgtctcatcatatatatatgctaattaatggatgtgtgtcctcttcacttcaattccttctttcctttcctatgctttgagatc

SEQ ID NO: 24 - DNA, 4714 bp, Artificial Sequence
  RAB17 TPS7a Ubi4_MAR
aaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatctctccaccatggtctccaagtcctactctaatctgctcgatctcacctccggcgatgggttcgatttccgccagcccttcaagtccctgccacgcgtggtcacctccccaggcatcattagcgatacagactgggatactatctccgacggcgattcagtggggtccgcttccagcacggagcggaagatcattgtcgccaatttcctgcctctcaactgcacccgcgacgagaccggcgtgctgtcgttctctctcgaccacgatgcgctcctgatgcagctgaaggattcattctccaacgagacagacgttgtctacgtgggcagcctcaaggttcaggtggatccaggggagcaggaccaggtggcccagaagctcctgagggagtaccggtgcatcccgaccttcctgccctcggacctccagcagcagttctaccacggcttctgcaagcagcagctgtggccactcttccattacatgctgcctatctgcctcgataagggcgagctgttcgaccgcagcctgttccaggcctacgtcagggcgaataagctgttcgccgataaggttatggaggctatcaacgccgacgatgacttcgtctgggttcacgactaccatctgatgctcctgccgaccttcctgaggaagcggctccaccgcatcaagattggcttcttcctccattccccgttcccctcgtctgagatctaccgcacactccccgtcagggacgagattctgaagtccctcctgaacgccgatctcatcggcttccagactttcgactacgccaggcacttcctgagctgctgctcgcgcctcctgggcctccattacgagagcaagcggggctacatcgggattgagtacttcgggcgcacagtctcgctgaagatcctctctgtgggcgtccacattgggcggctggagtcggtgctgaagctcccagcgactgtgtctaaggtccaggagatcgagcagaggtacaagggcaagattctgatgctcggggtggatgacatggacatcttcaagggcatttccctcaagttcctgggcctggagctcctcctcgaccggaatccgaagctccgcgagaaggtcgttctggtccagatcattaacccggctaggtctacgggcaaggacgtgcaggaggctattacggaggccgtttcagtggcggagaggatcaacaccaattacggctcatccagctacaagccagtggtcctgatcgaccaccatattcctttctacgagaagattgcgttctacgccgcgtccgattgctgcatcgttaatgctgtgcgcgacggcatgaacctcgtcccgtacgagtacaccgtttgcaggcaggggaatgaggagattgataagctcaggggcctggggaaggacacccaccatacatcaactctgatcgtgtccgagttcgtgggctgctccccctccctctctggggcgttcagggtcaacccctggtccgtcgatgacgttgcggatgccctctgcagggctacggacctgacggagtccgagaagcgcctgaggcacgagaagcattaccgctacgtgtccacgcacgacgtggcgtactgggctaggtccttcgcgcaggacctggagcgggcttgcaaggaccattacagccgcaggtgctgggctatcggcttcgggctcaacttccgcgtcattgccctgtcgcccggcttcaggaagctctcgtctgagcacttcgtgtcatcctacaataaggcctcccgccgcgccatcttcctcgactacgatggcaccctggtgccgcagagctcgatcaacaaggcccccagcgaggaggtcatctcggtcctgaacacgctctgcaatgacccgaagaacatcgttttcattgtgtccggccgggggcgcgatagcctggacgagtggttctccccgtgcgagaagctgggcctcgctgctgagcacgggtacttcatccgctggagcaaggaggcggcttgggagtcttcatactcccgcccgcagcaggagtggaagcacatcgcggagcctgtcatgcaggtttacacggagaccacggacggctccagcatcgagtctaaggagtcagccctcgtctggcactacctggacgcggatcatgacttcggctcgttccaggctaaggagctccaggggcacctggagcgcgtgctctctaatgagccagttgtggtcaagtgcggccattacatcgtcgaggttaagccgcagggcgtgagcaaggggctggcggtcaataagctcatccacaccctggttaagaacggcaaggccccggatttcctcatgtgcgtggggaacgaccgctccgatgaggacatgttcgagagcatcaatggcatgacatctaacgccgtcctgtcaccaactatgcctgagctgttcgcctgctctgtgggccagaagccatcaaaggctaagtactacgttgatgacacatccgaggtcatccgcctgctcaagaacgtgactaggattcctagccagcggcaggatgtgtcggcgagccacgggcgggtcactttccggggcgttctggactacgttgattagaactcctgggccatgaagctgtccttccaggttcacaagtctggtgccttcttctgtccctccgatggagattatctgcatgtcgtggtcgtgtcctgatcgaatcctcgttgaatccctatgtttttcttcaagaaatgtgagtcctatgtcagtctggttgcgtttgtgaacatttctgctgctgagcagcactttggctggaactgtgcaatgaaataaatggaaccctggtttctggttatgtgtgtgttagctaatgtttttgaagtggaagctctaatcttctatcgcgttgctactacaattctgcttgtgttttgatgttcttggtttctgttagttggttcagaggaagttttgcttccacagactaagatgcagttgaactttggttgccctggtttctagatttcatttgtgctggttgagtgatagtaagaaacaaccggtgttcacatataatcaggttttgtgctgctcgagtgatcgtcaaaaaccaccggtgttcacatctaaaaaggtttcgatccccaggtttagatctcccgtttaattccaaaaaaaaagttctgtgtacttgcatttagttgggtggttgatgctggaaagagtaactttcaagagtaataatctttggtgactactctgtttcaactgatcaatccctaggaaaggtacacctttacttagggaagaaattcttagaaccttgcactttgtttcaactgataatagtatactttattagataaaaaatattcagatatattagacaccggatgtcatccactcatccttacaaacctctgtcatggtcctgcagaaatgtttgccagctccagtggcttcctgataaatctgtggagtgcctgttaatcggctgccaatttttgctgagcactgtatatatgttagtaagtactattgggccaccaattcgattttgacacagcactattggtccaccaattcgattctgacacagcactgcataatttgaaacgtgttgctccattttgcaaggctacaaatttagatcatgtttagcattctgtgggatacaatatatggatatcgaacaaacttggtatgtcagagaaaaaatagtttattttcaaaactaacatttttaaagccttctatgaactttaaaccttcagcatttgggatcaagatgagtgctcgaacaagagtgcactttttctccaaaataatctactacagagttcttttttatatataaaaaaacttatacttaacagataaatcagactttttctgctccatatcaccttgacaaatcaaagaagcagcaccagcgaagggtattattattgaggtaaatataagatctcgtttactgaaaaagaccgcgtgtttacctaaactaccattttgctttgatagcagcatacatgtgatagaattgcggatcctaccgtgctgactgtgaaggtggtaggggtgagagattggtgggcgaggtctgaacgagcgagaacagtactgcatttactgttcacaaggaggcggcttaggttttgggtctcccagctctctaagggaagctgagaattatgattctcttgcttaattatttcttaaccaaagttataaatatatagcctatgagatcctaatttatggaaataactaaacta